(19) United States
(12) Patent Application Publication
(10) Pub. No.: US 2014/0052053 A1
(43) Pub. Date: Feb. 20, 2014

(54) Infusion Sleeve with Motion Reduction Profile

(71) Applicant: Alcon Research, Ltd., (US)

(72) Inventors: Karen T. Hong, Corona, CA (US); Rudolph Zacher, Costa Mesa, CA (US); Kristine Velasco, Foothill Ranch, CA (US)

(73) Assignee: ALCON RESEARCH, LTD., Fort Worth, TX (US)

(21) Appl. No.: 13/750,041

(22) Filed: Jan. 25, 2013

Related U.S. Application Data
(63) Continuation-in-part of application No. 13/456,353, filed on Apr. 26, 2012.

(51) Int. Cl. A61F 9/007; A61M 3/02

(52) U.S. Cl. CPC A61F 9/00745; A61M 3/0279
USPC 604/22; 604/525

(57) ABSTRACT

An infusion sleeve has a flexible tube enclosing a lumen. The tube has a plurality of wall segments, each wall segment located between the lumen and an exterior surface of the tube and extending parallel to a central axis of the tube. The plurality of wall segments includes at least two thick wall segments and at least two thin wall segments alternately arranged such that each thick wall segment is adjacent to two thin wall segments, and each thin wall segment is adjacent to two thick wall segments.

This application is a continuation-in-part of U.S. application Ser. No. 13/456,353 filed Apr. 26, 2012.

### BACKGROUND OF THE INVENTION

The present invention relates to phacoemulsification surgery and more particularly to an infusion sleeve that reduces the likelihood of injury to delicate eye structures during surgery.

The human eye functions to provide vision by transmitting light through a clear outer portion called the cornea, and focusing the image by way of a crystalline lens onto a retina. The quality of the focused image depends on many factors including the size and shape of the eye, and the transparency of the cornea and the lens. When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. A typical surgical hand piece suitable for phacoemulsification procedures consists of an ultrasonically driven phacoemulsification hand piece, an attached hollow cutting needle surrounded by an irrigation sleeve, and an electronic control console. The hand piece assembly is attached to the control console by an electric cable and flexible tubing. Through the electric cable, the console varies the power level transmitted by the hand piece to the attached cutting needle. The flexible tubing supplies irrigation fluid to the surgical site and draws aspiration fluid from the eye through the hand piece assembly.

The operative part in a typical hand piece is a centrally located, hollow resonating bar or horn directly attached to a set of piezoelectric crystals. The crystals supply the required ultrasonic vibration needed to drive both the horn and the attached cutting needle during phacoemulsification, and are controlled by the console. The crystal/horn assembly is suspended within the hollow body or shell of the hand piece by flexible mountings. The hand piece body terminates in a reduced diameter portion or nosecone at the body's distal end. Typically, the nosecone is externally threaded to accept the hollow irrigation sleeve, which surrounds most of the length of the cutting needle. Likewise, the horn bore is internally threaded at its distal end to receive the external threads of the cutting tip. The irrigation sleeve also has an internally threaded bore that is screwed onto the external threads of the nosecone. The cutting needle is adjusted so that its tip projects only a predetermined amount past the open end of the irrigation sleeve.

During the phacoemulsification procedure, the tip of the cutting needle and the end of the irrigation sleeve are inserted into the anterior capsule of the eye through a small incision in the outer tissue of the eye. The surgeon brings the tip of the cutting needle into contact with the lens of the eye, so that the vibrating tip fragments the lens. The resulting fragments are aspirated out of the eye through the interior bore of the cutting needle, along with irrigation solution provided to the eye during the procedure, and into a waste reservoir.

Throughout the procedure, irrigating fluid is introduced into the eye, passing between the irrigation sleeve and the cutting needle and exiting into the eye at the tip of the irrigation sleeve and/or from one or more ports, or openings, in the irrigation sleeve near its end. The irrigating fluid protects the eye tissues from the heat generated by the vibrating of the ultrasonic cutting needle. Furthermore, the irrigating fluid suspends the fragments of the emulsified lens for aspiration from the eye.

Power is applied to the hand piece to vibrate the cutting needle. In general, the amplitude of needle movement (or vibration) is proportional to the power applied. In conventional phacoemulsification systems, the needle vibrates back and forth producing a longitudinal needle stroke. In improved systems, the needle may be caused to vibrate in a twisting or torsional motion. Regardless of the type of vibration, the magnitude of vibration (or amplitude of needle stroke) varies with applied power.

One complication that may arise during the procedure is damage to eye structures such as the iris. As the needle vibrates torsionally, it imparts circumferential motion to the irrigation sleeve. The circumferential vibrations transmitted by the sleeve to an eye structure, such as the iris, may damage it. An improved irrigation sleeve may be used to decrease the physical force transmitted by circumferential motion of the sleeve to eye structures.

### SUMMARY OF THE INVENTION

In one embodiment consistent with the principles of the present invention, the present invention is an infusion sleeve has a flexible tube enclosing a lumen. The tube has a plurality of wall segments, each wall segment located between the lumen and an exterior surface of the tube and extending parallel to a central axis of the tube. The plurality of wall segments includes at least two thick wall segments and at least two thin wall segments alternately arranged such that each thick wall segment is adjacent to two thin wall segments, and each thin wall segment is adjacent to two thick wall segments.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a diagram of the components in the fluid path of a phacoemulsification system.

FIGS. 2A-2C are perspective views of the distal end of a phacoemulsification needle and irrigation sleeve according to the principles of the present invention.

FIGS. 3A-3C are cross section views of a prior art infusion sleeve.

FIGS. 4A-4C are cross section views of an infusion sleeve according to the principles of the present invention.

FIGS. 5A-5C are cross section views of an infusion sleeve according to the principles of the present invention.

FIGS. 6A-6C are cross section views of an infusion sleeve according to the principles of the present invention.

FIGS. 7A-7C are cross section views of an infusion sleeve according to the principles of the present invention.

FIG. 8 is a cross section view of an infusion sleeve according to the principles of the present invention.

FIG. 9 is a cross section view of an infusion sleeve according to the principles of the present invention.

FIG. 10 is a cross section view of an infusion sleeve according to the principles of the present invention.

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

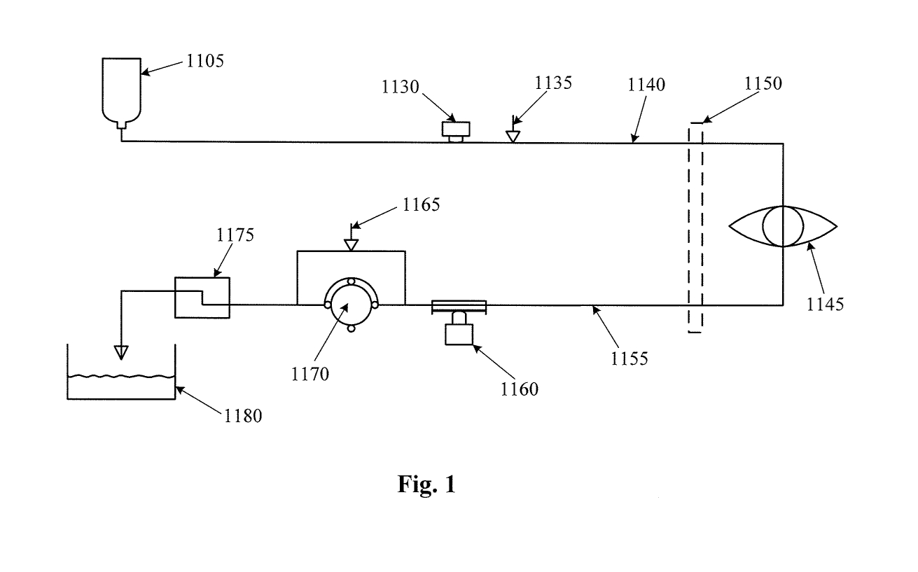

In one embodiment of the present invention, FIG. 1 is a diagram of the components in the fluid path of a phacoemulsification system. FIG. 1 depicts the fluid path through the eye 1145 during cataract surgery. The components include an irrigation fluid source 1105, an irrigation pressure sensor 1130, an irrigation valve 1135, an irrigation line 1140, a hand piece 1150, an aspiration line 1155, an aspiration pressure sensor 1160, a vent valve 1165, a pump 1170, a reservoir 1175 and a drain bag 1180. The irrigation line 1140 provides irrigation fluid to the eye 1145 during cataract surgery. The aspiration line 1155 removes fluid and emulsified lens particles from the eye during cataract surgery.

When irrigation fluid exits irrigation fluid source 1105, it travels through irrigation line 1140 and into the eye 1145. An irrigation pressure sensor 1130 measures the pressure of the irrigation fluid in irrigation line 1140. An optional irrigation valve 1135 is also provided for on/off control of irrigation. Irrigation pressure sensor 1130 is implemented by any of a number of commercially available fluid pressure sensors and can be located anywhere in the irrigation fluid path (anywhere between the irrigation source 1105 and the eye 1145).

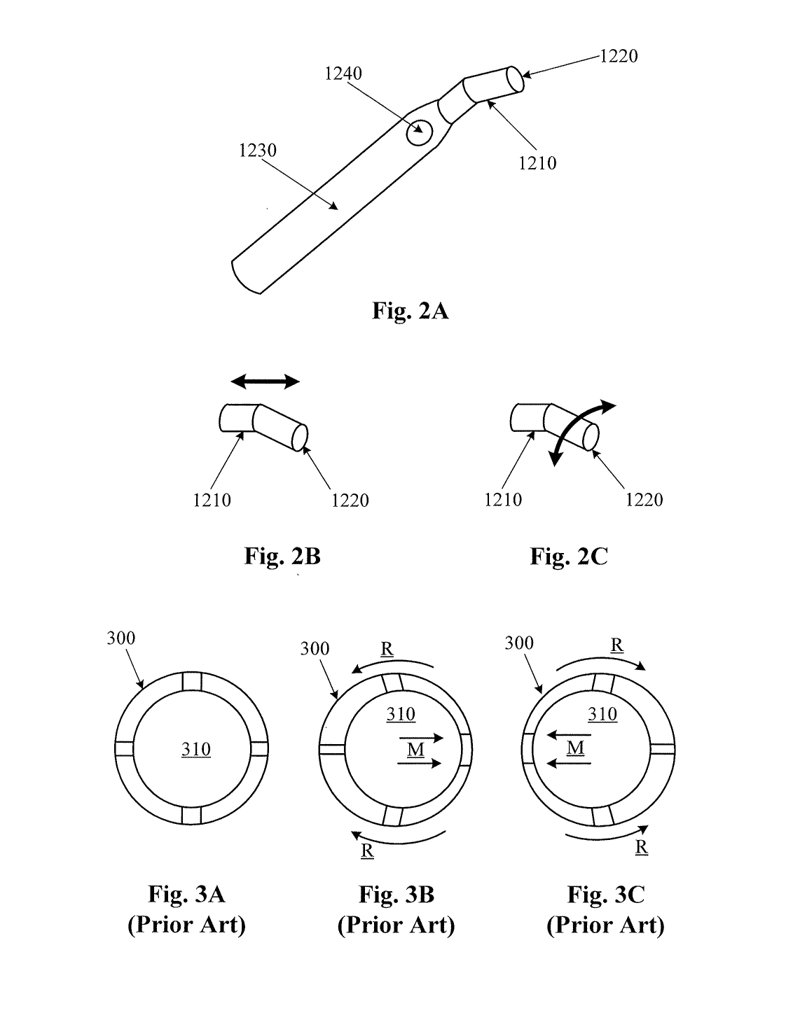

A hand piece 1150 is placed in the eye 1145 during a phacoemulsification procedure. The hand piece 1150 has a hollow needle (as seen in FIG. 2) that is ultrasonically vibrated in the eye to break up the diseased lens. A sleeve located around the needle provides irrigation fluid from irrigation line 1140. The irrigation fluid passes through the space between the outside of the needle and the inside of the sleeve (as more clearly shown in FIG. 2A). Fluid and lens particles are aspirated through the hollow needle. In this manner, the interior passage of the hollow needle is fluidly coupled to aspiration line 1155. Pump 1170 draws the aspirated fluid from the eye 1145. An aspiration pressure sensor 1160 measures the pressure in the aspiration line. An optional vent valve can be used to vent the vacuum created by pump 1170. The aspirated fluid passes through reservoir 1175 and into drain bag 1180.

FIG. 2A is a perspective view of the distal end of a phacoemulsification hand piece according to the principles of the present invention. In FIG. 2, a phacoemulsification needle 1210 is surrounded by an irrigation sleeve 1230. The phacoemulsification needle 1210 has an open end 1220 through which lens particles are aspirated from the eye during cataract surgery. The irrigation sleeve 1230 has an optional opening 1240 through which irrigation fluid flows into the eye. The needle 1210 and sleeve 1230 are both inserted into the anterior chamber of the eye during cataract surgery. When power is applied to the hand piece, the needle 1210 vibrates ultrasonically. This is more clearly seen in FIGS. 2B and 2C. In FIG. 2B, needle 1210 vibrates in longitudinal mode (back and forth). In FIG. 2C, needle 1210 vibrates in torsional mode (or in a twisting or sweeping manner).

The two different modes (longitudinal and torsional) produce two different needle motions as shown in FIGS. 2B and 2C. In general, longitudinal mode can act to cut a cataractous lens by impacting the end of the needle 1210 against the lens much like a jackhammer. Torsional mode can act to cut a lens with a side to side sweep of the end of the needle 1210. Depending on the needle geometry, the twisting motion imparted to the needle 1210 in torsional mode generally produces a side to side sweep of the end of the needle 1210. In other instances, the end of the needle 1210 sweeps in an arc. Regardless, torsional mode may be more effective in cutting a lens because it allows aspiration through open end 1220 of needle 1210 to hold the lens material on the needle 1210 for more effective cutting. In addition, in torsional mode, each sweep of the needle 1210 acts to cut the lens. In contrast, longitudinal mode produces a jack hammer motion that impacts the lens only in a forward direction (and not in a return direction). Moreover, longitudinal mode may act to repel the lens material away from the needle which may reduce cutting efficiency.

The effect of the sweeping motion of needle 1210 on the irrigation sleeve is shown in FIGS. 3A-3C. FIGS. 3A-3C are cross section views of a prior art infusion sleeve. A needle would occupy the lumen 310 of sleeve 300. As shown in FIG. 3A, sleeve 300 has a generally circular cross section as does the lumen 310 bounded by sleeve 300. In this manner, sleeve 300 is generally cylindrical or tube shaped with an interior passage or lumen 310 that has a circular cross section. In FIGS. 3A-3C, the boxes on the sleeve wall located at twelve, three, six, and nine o'clock are for illustrating the sleeve movement seen in FIGS. 3B and 3C.

As shown in FIGS. 3B and 3C, when a needle (not shown) located in lumen 310 is vibrated torsionally or in a sweeping manner (needle motion denoted by “M”), a circumferential, radial or rotating motion is imparted to sleeve 300 (sleeve motion is denoted by “R”). Needle motion M alternately compresses each side of the wall of sleeve 300 while expanding the other side of the wall of sleeve 300. The top and bottom walls of sleeve 300 generally move circumferentially in an arc R. In this manner, torsional vibration of the needle (not shown) in lumen 310 causes significant motion of the sleeve 300. Force is transmitted from the needle to the sleeve 300 in the direction of needle motion M resulting in a compression of a side wall of sleeve 300 as shown. In addition, the walls of sleeve 300 (top and bottom walls shown in FIGS. 3B and 3C) move circumferentially around the needle. Such motion may damage eye structures such as the iris.

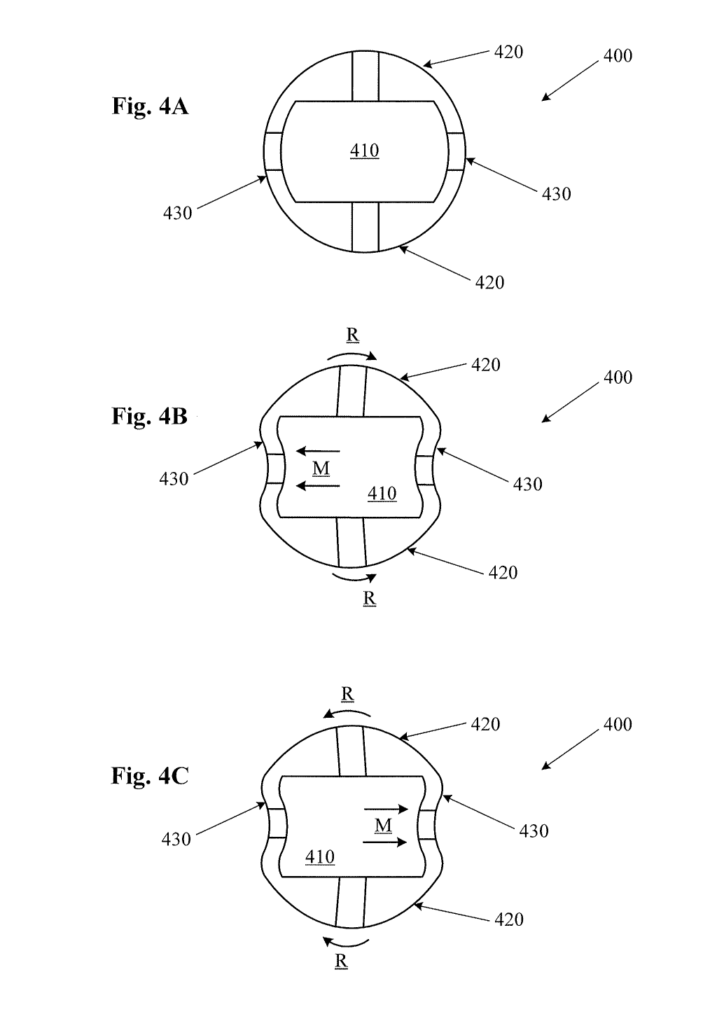

FIGS. 4A-4C are cross section views of an infusion sleeve according to the principles of the present invention. In FIG. 4A, sleeve 400 has an internal lumen 410, two thick walls 420, and two thin walls 430. Lumen 410 has an oblong cross section, although other cross sections, such as an elliptical cross section, may also be employed. The needle would be located in lumen 410. The exterior of the sleeve 400 has a generally circular cross section and is in the shape of a tube. In this example, two thick walls 420 are located at twelve and six o'clock, and two thin walls 430 are located at three and nine o'clock. In FIGS. 4A-4C, the boxes on the sleeve wall located at twelve, three, six, and nine o'clock are for illustrating the sleeve movement seen in FIGS. 4B and 4C.

While the location of thick walls 420 and thin walls 430 are shown at twelve and six o'clock and at three and nine o'clock, respectively, in other embodiments of the present invention, thick walls 420 and thin walls 430 may be located at any point on the sleeve as long as they are alternated. In other words, as one travels around the periphery of sleeve 400, one would encounter a thick wall 420 followed by a thin wall 430, followed by a thick wall 420, etc. Any number of thick walls 420 and thin walls 430 may be employed. In addition, the thin walls 430 may not be of a uniform cross section, but instead may transition gradually into the cross section of the thick walls 420. In this manner, the thick walls 420 and thin walls 430 may have cross sections that vary along their lengths. The thin walls 430 may also have lengths that are greater than, less than, or the same as the thick walls 430.

As shown in FIGS. 4B and 4C, when a needle (not shown) located in lumen 410 is vibrated torsionally or in a sweeping manner (needle motion denoted by “M”), a much smaller circumferential or rotating motion is imparted to the thick walls 420 of sleeve 400 (thick wall 420 motion is denoted by “R”). Needle motion M alternately deforms each thin wall 430. The thick walls 420 of sleeve 400 generally move very slightly circumferentially in an arc R. In general, the thin walls 430 are deformable such that little circumferential motion is imparted to the thick walls 420. Moreover, deformation of thin walls 430 also imparts very little force to adjacent eye structures. As such, the improved sleeve design of FIG. 4A reduces the force applied to eye structures by the sleeve 400 when in use.

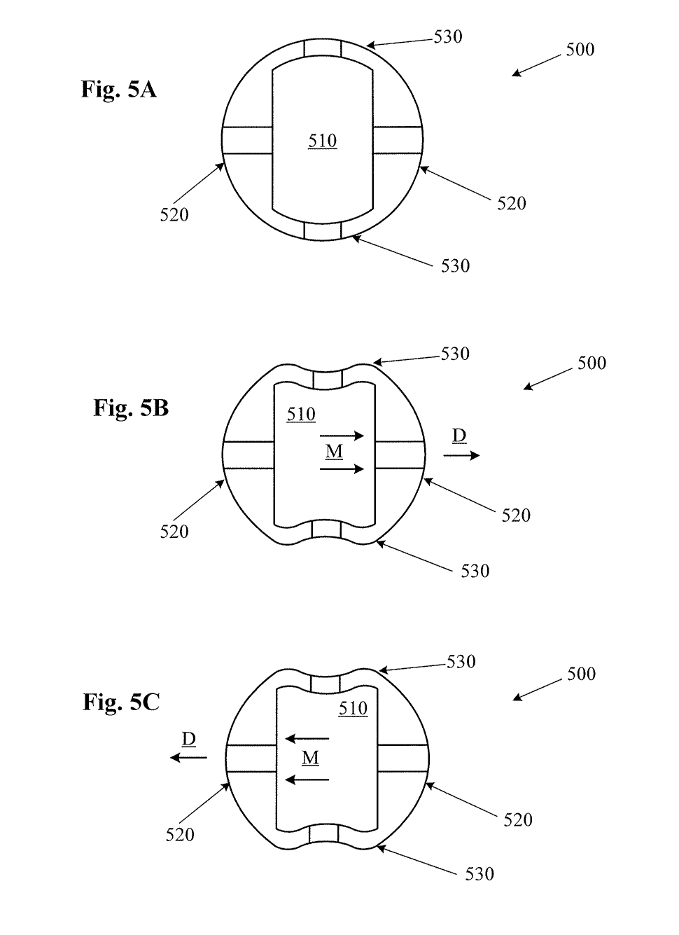

FIGS. 5A-5C are cross section views of an infusion sleeve according to the principles of the present invention. In FIG. 5A, sleeve 500 has an internal lumen 510, two thick walls 520, and two thin walls 530. Lumen 510 has an oblong cross section, although other cross sections, such as an elliptical cross section, may also be employed. The needle would be located in lumen 510. The exterior of the sleeve 500 has a generally circular cross section and is in the shape of a tube. In this example, two thick walls 520 are located at three and nine o'clock, and two thin walls 530 are located at twelve and six o'clock. In FIGS. 5A-5C, the boxes on the sleeve wall located at twelve, three, six, and nine o'clock are for illustrating the sleeve movement seen in FIGS. 5B and 5C.

While the location of thick walls 520 and thin walls 530 are shown at three and nine o'clock and at twelve and six o'clock, respectively, in other embodiments of the present invention, thick walls 520 and thin walls 530 may be located at any point on the sleeve as long as they are alternated. In other words, as one travels around the periphery of sleeve 500, one would encounter a thick wall 520 followed by a thin wall 530, followed by a thick wall 520, etc. Any number of thick walls 520 and thin walls 530 may be employed. In addition, the thin walls 530 may not be of a uniform cross section, but instead may transition gradually into the cross section of the thick walls 520. In this manner, the thick walls 520 and thin walls 530 may have cross sections that vary along their lengths. The thin walls 530 may also have lengths that are greater than, less than, or the same as the thick walls 530.

As shown in FIGS. 5B and 5C, when a needle (not shown) located in lumen 510 is vibrated torsionally or in a sweeping manner (needle motion denoted by “M”), small linear motion is imparted to the thick walls 520 of sleeve 500 (thick wall 520 motion is denoted by “D”). Needle motion M alternately deforms each thin wall 530, much as the thin walls 430 of FIGS. 4B and 4C are deformed. The thick walls 520 of sleeve 500 generally move very slightly to and fro in a linear manner D. In general, the thin walls 530 are deformable such that little motion is imparted to the thick walls 520. Moreover, deformation of thin walls 530 also imparts very little force to adjacent eye structures. As such, the improved sleeve design of FIG. 5A reduces the force applied to eye structures by the sleeve 500 when in use.

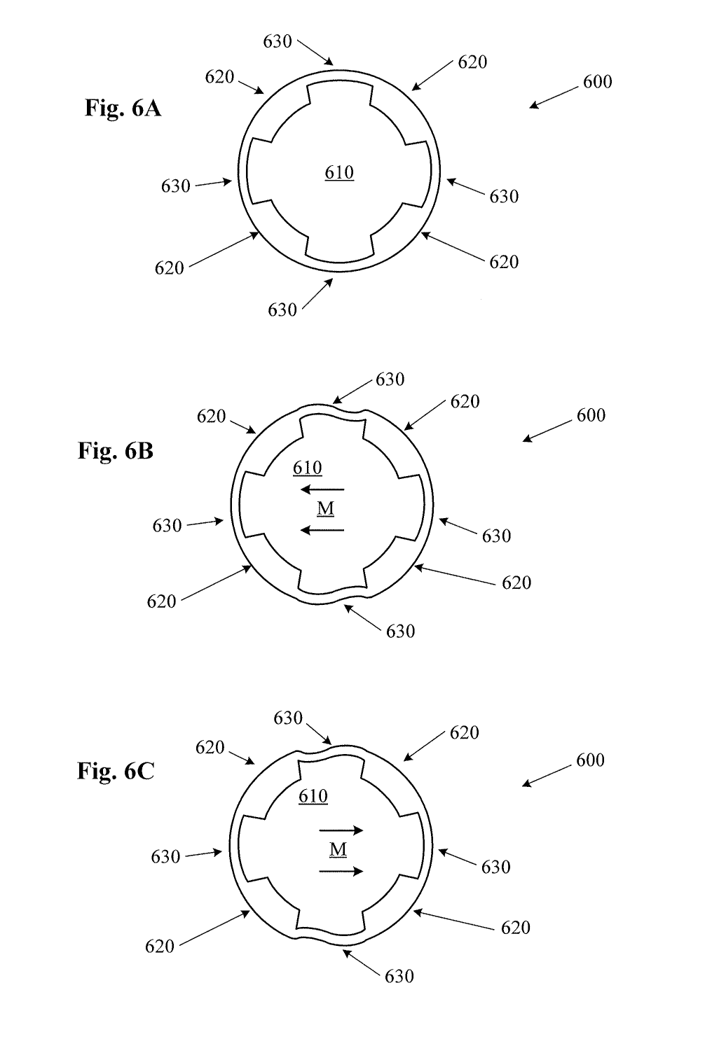

FIGS. 6A-6C are cross section views of an infusion sleeve according to the principles of the present invention. In FIG. 6A, sleeve 600 has an internal lumen 610, four thick walls 620, and four thin walls 630. Lumen 610 has a sprocket-type cross section, although other cross sections, such as a star-shaped cross section, may also be employed. The needle would be located in lumen 610. The exterior of the sleeve 600 has a generally circular cross section and is in the shape of a tube. In this example, four thin walls 630 are located at twelve, three, six, and nine o'clock. The four thick walls 620 are located adjacent to the four thin walls 630.

In other embodiments of the present invention, thick walls 620 and thin walls 630 may be located at any point on the sleeve as long as they are alternated. In other words, as one travels around the periphery of sleeve 600, one would encounter a thick wall 620 followed by a thin wall 630, followed by a thick wall 620, etc. Any number of thick walls 620 and thin walls 630 may be employed. In addition, the thin walls 630 may not be of a uniform cross section, but instead may transition gradually into the cross section of the thick walls 620. In this manner, the thick walls 620 and thin walls 630 may have cross sections that vary along their lengths. The thin walls 630 may also have lengths that are greater than, less than, or the same as the thick walls 630.

As shown in FIGS. 6B and 6C, when a needle (not shown) located in lumen 610 is vibrated torsionally or in a sweeping manner (needle motion denoted by “M”), small linear motion is imparted to the thick walls 620 of sleeve 600. Needle motion M alternately deforms thin wall 630, much as the thin walls 430 of FIGS. 4B and 4C are deformed. The thick walls 620 of sleeve 600 generally move very slightly to and fro in a linear manner. In general, the thin walls 630 are deformable such that little motion is imparted to the thick walls 620. In FIGS. 6B and 6C, the thin walls 630 located at 6 and 12 o'clock are deformed slightly. In general, thin walls 630 may be compressed or stretched slightly depending on the movement M of the needle (not shown). Moreover, deformation of thin walls 630 also imparts very little force to adjacent eye structures. As such, the improved sleeve design of FIG. 6A reduces the force applied to eye structures by the sleeve 600 when in use.

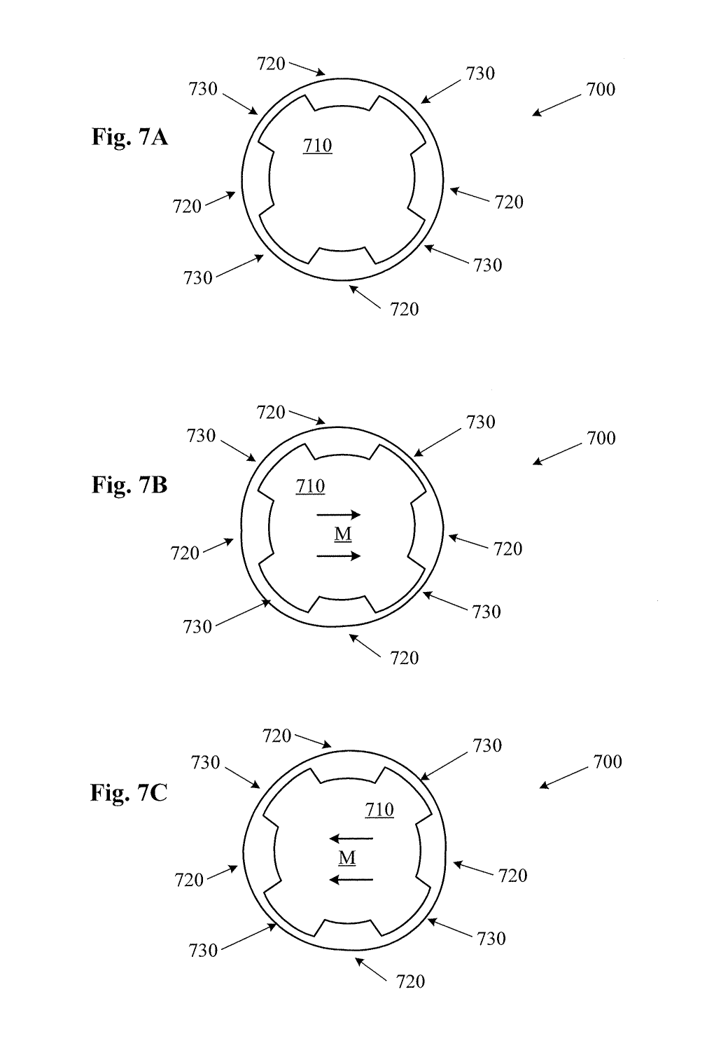

FIGS. 7A-7C are cross section views of an infusion sleeve according to the principles of the present invention. In FIG. 7A, sleeve 700 has an internal lumen 710, four thick walls 720, and four thin walls 730. Lumen 710 has a sprocket-type cross section, although other cross sections, such as a star-shaped cross section, may also be employed. The needle would be located in lumen 710. The exterior of the sleeve 700 has a generally circular cross section and is in the shape of a tube. In this example, four thick walls 720 are located at twelve, three, six, and nine o'clock. The four thin walls 730 are located adjacent to the four thick walls 720.

In other embodiments of the present invention, thick walls 720 and thin walls 730 may be located at any point on the sleeve as long as they are alternated. In other words, as one travels around the periphery of sleeve 700, one would encounter a thick wall 720 followed by a thin wall 730, followed by a thick wall 720, etc. Any number of thick walls 720 and thin walls 730 may be employed. In addition, the thin walls 730 may not be of a uniform cross section, but instead may transition gradually into the cross section of the thick walls 720. In this manner, the thick walls 720 and thin walls 730 may have cross sections that vary along their lengths. The thin walls 730 may also have lengths that are greater than, less than, or the same as the thick walls 730.

As shown in FIGS. 7B and 7C, when a needle (not shown) located in lumen 710 is vibrated torsionally or in a sweeping manner (needle motion denoted by “M”), small linear motion is imparted to the thick walls 720 of sleeve 700. Needle motion M alternately deforms thin wall 730, much as the thin walls 430 of FIGS. 4B and 4C are deformed. The thick walls 720 of sleeve 700 generally move very slightly to and fro in a linear manner. In general, the thin walls 730 are deformable such that little motion is imparted to the thick walls 720. In general, thin walls 730 may be compressed or stretched slightly depending on the movement M of the needle (not shown). Moreover, deformation of thin walls 730 also imparts very little force to adjacent eye structures. As such, the improved sleeve design of FIG. 7A reduces the force applied to eye structures by the sleeve 700 when in use.

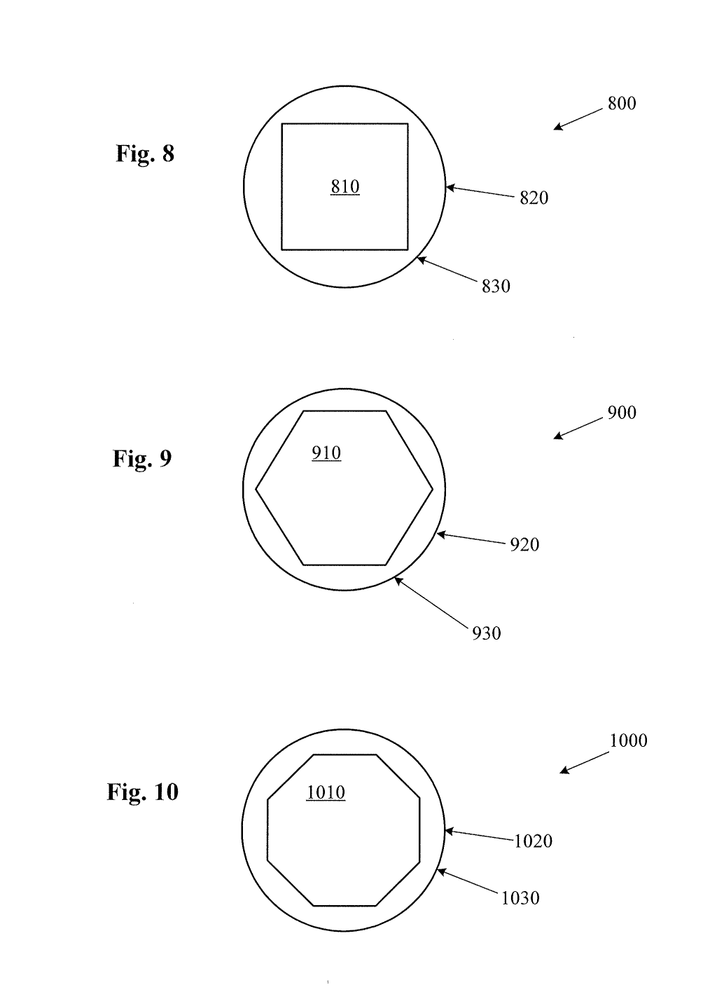

FIG. 8 is a cross section view of an infusion sleeve according to the principles of the present invention. In FIG. 8, sleeve 800 has an internal lumen 810, four thick walls 820, and four thin walls 830. Lumen 810 has a square cross section. The needle would be located in lumen 810. The exterior of the sleeve 800 has a generally circular cross section and is in the shape of a tube. In this example, four thick walls 820 are located at twelve, three, six, and nine o'clock. The four thin walls 830 are located adjacent to the four thick walls 820. In addition, the thin walls 830 are not of a uniform cross section, but instead transition gradually into the cross section of the thick walls 820. In this manner, the thick walls 820 and thin walls 830 have cross sections that vary along their lengths. The thin walls 830 may also have lengths that are greater than, less than, or the same as the thick walls 830.

FIG. 9 is a cross section view of an infusion sleeve according to the principles of the present invention. In FIG. 9, sleeve 900 has an internal lumen 910, six thick walls 920, and six thin walls 930. Lumen 910 has a hexagonal cross section. The needle would be located in lumen 910. The exterior of the sleeve 900 has a generally circular cross section and is in the shape of a tube. In this example, the six thin walls 930 are located adjacent to the six thick walls 920. In addition, the thin walls 930 are not of a uniform cross section, but instead transition gradually into the cross section of the thick walls 920. In this manner, the thick walls 920 and thin walls 930 have cross sections that vary along their lengths. The thin walls 930 may also have lengths that are greater than, less than, or the same as the thick walls 930.

FIG. 10 is a cross section view of an infusion sleeve according to the principles of the present invention. In FIG. 10, sleeve 1000 has an internal lumen 1010, eight thick walls 1020, and eight thin walls 1030. Lumen 1010 has an octagonal cross section. The needle would be located in lumen 1010. The exterior of the sleeve 1000 has a generally circular cross section and is in the shape of a tube. In this example, the eight thin walls 1030 are located adjacent to the eight thick walls 1020. In addition, the thin walls 1030 are not of a uniform cross section, but instead transition gradually into the cross section of the thick walls 1020. In this manner, the thick walls 1020 and thin walls 1030 have cross sections that vary along their lengths. The thin walls 1030 may also have lengths that are greater than, less than, or the same as the thick walls 1030.

The sleeves 400, 500, 600, 700, 800, 900, and 1000 depicted in FIGS. 4A-4C, 5A-5C, 6A-6C, 7A-7C, 8, 9, and 10 are made of an elastic material such as silicone or other suitable polymer. As such, the sleeves 400, 500, 600, 700, 800, 900, and 1000 are flexible and can deform as shown in FIGS. 4A-4C, 5A-5C, 6A-6C, 7A-7C, 8, 9, and 10. The sleeves 400, 500, 600, 700, 800, 900, and 1000 may also be described as generally flexible tubes. In addition, the cross section views shown in FIGS. 4A-4C, 5A-5C, 6A-6C, 7A-7C, 8, 9, and 10 may represent the sleeve at any point or at particular points along the needle that is inserted into the eye. The sleeves 400, 500, 600, 700, 800, 900, and 1000 may have the same or a different cross section at a location that is not inserted into the eye (for example, at a location further posterior the end of the needle). For example, the distal one third of the sleeve may have a cross section shown in FIGS. 4A-4C, 5A-5C, 6A-6C, 7A-7C, 8, 9, and 10, while the proximal two thirds may have a different cross section (such as the cross section of a simple flexible tube without thick and thin segments). In another example, the sleeve has the same cross section along the entire length of the needle. Other combinations of cross sections along the length of the sleeve may also be employed.

From the above, it may be appreciated that the present invention provides an improved irrigation sleeve for phacoemulsification surgery. The present invention provides an irrigation sleeve with thick wall and thin wall segments that decrease the amount of motion transferred to adjacent eye structures when a needle located in the lumen of the sleeve is vibrated torsionally. The present invention is illustrated herein by example, and various modifications may be made by a person of ordinary skill in the art.

Other embodiments of the invention will be apparent to those skilled in the art from consideration of the specification and practice of the invention disclosed herein.

It is intended that the specification and examples be considered as exemplary only, with a true scope and spirit of the invention being indicated by the following claims.

What is claimed is:

1. An irrigation sleeve comprising:
a flexible tube enclosing a lumen, the tube having a plurality of thick and thin wall segments, each thick and thin wall segment located between the lumen and an exterior surface of the tube;
wherein the thick and thin wall segments are arranged in an alternating manner around the sleeve.

2. The irrigation sleeve of claim 1 wherein the plurality of thin wall segments are deformable.

3. The irrigation sleeve of claim 1 wherein the cross section of the lumen is selected from the group consisting of: an oblong shape, an ellipse, a square, a hexagon, an octagon, a polygon, a sprocket-type shape, and a star-type shape.

4. The irrigation sleeve of claim 1 wherein an exterior of the flexible tube has a generally circular cross section

5. The irrigation sleeve of claim 1 wherein the lumen holds a phacoemulsification needle.

6. The irrigation sleeve of claim 6 wherein when the phacoemulsification needle is vibrated torsionally, very little circumferential motion is imparted to the irrigation sleeve.

7. The irrigation sleeve of claim 1 wherein the thin wall segments are deformable.

8. The irrigation sleeve of claim 7 wherein when motion is imparted to the sleeve, the thick wall segments move and the thin wall segments deform.

9. The irrigation sleeve of claim 8 wherein deformation of the thin walls absorbs energy thus preventing that energy from being transmitted to an eye structure.
